(19) United States
(12) Patent Application Publication
(10) Pub. No.: US 2008/0050323 A1
(43) Pub. Date: Feb. 28, 2008

(54) Sunscreen composition

(75) Inventors: Prasenjit Bhunia, West Bengal (IN); Amitava Pramanik, Bangalore (IN); Srinivasa Gopalan Raman, Bangalore (IN)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc. d/b/a UNILEVER

(21) Appl. No.: 11/895,220

(22) Filed: Aug. 23, 2007

(30) Foreign Application Priority Data
Aug. 23, 2006 (IN) 1323/MUM/06
Mar. 7, 2007 (EP) EP07103667

(51) Int. Cl. A61K 8/35; A61K 8/36; A61Q 17/04

(52) U.S. Cl. 424/59

(57) ABSTRACT

It has been reported that many organic sunscreens lose their efficacy on exposure to UV rays. Further some of the organic sunscreens are known to interact with each other when applied on the skin with or without exposure to UV radiation. Thus such compositions are not stable. They are not very effective in blocking the UV-rays a few minutes or hours after the product has been applied to the skin. Further when such compositions are used and then exposed to sunlight or UV rays, the period over which the skin is actually protected is very short, while the consumer is under the mistaken impression that the composition is protecting his/her skin. This false impression can lead to the consumer exposing himself/herself more to sunlight thereby getting more damage to the skin.

A solution to the foregoing problem is provided by a stable sunscreen composition comprising:
(a) a complex of a first organic sunscreen % with a first paramagnetic metal ion; and
(b) a second organic sunscreen.

The invention relates to sunscreen compositions. The invention more particularly relates to sunscreen compositions containing organic sunscreens that are so modified as to display enhanced stability.

Sunscreens have been used in cosmetic compositions for topical application for a long time. Sunscreens are added to skin cosmetic compositions to block the ultra-violet (UV) radiation of the sun from affecting skin exposed to sunlight. Inorganic sunscreens act by physically blocking some or all of the UV rays by causing the rays to be reflected or scattered away from the skin surface. Organic sunscreens act by absorbing some or all of the UV radiation thereby ensuring that the UV rays do not reach the skin surface. UV rays are generally classified as UV-A, UV-B or UV-C rays. UV-A generally covers the UV wavelength range from 320 to 400 nm, UV-B covers the 290 to 320 nm range and UV-C covers the 200 to 290 nm range. While damage to the skin due to exposure to UV-A rays have been reported to cause irritation, redness and sunburns, exposure to UV-B rays causes more severe irritation, redness and sunburns in addition to erythema. UV-C rays constitute a small percentage of sunlight as most of it is absorbed by the ozone layer. Hence sunscreen formulations have generally been formulated with a combination of UV-A and UV-B sunscreens.

It has been reported that many organic sunscreens lose their efficacy on exposure to UV rays. Further some of the organic sunscreens are known to interact with each other when applied on the skin with or without exposure to UV radiation. Thus such compositions are not stable. They are not very effective in blocking the UV-rays a few minutes or hours after the product has been applied to the skin. Further when such compositions are used and then exposed to sunlight or UV rays, the period over which the skin is actually protected is very short, while the consumer is under the mistaken impression that the composition is protecting his/her skin. This false impression can lead to the consumer exposing himself/herself more to sunlight thereby getting more damage to the skin.

There has been some efforts in improving the stability of sunscreens. It has been found that some classes of sunscreen when formulated with another class show improved stability. EP 780 119 (Givaudan, 1997) describes a photostable cosmetic light-screening composition comprising essentially a dibenzoylmethane type UV-A screening agent and a a-cyano-β,β-diphenyl acrylate stabiliser in specified amounts and ratios.

U.S. Pat. No. 6,436,375 (Sol-Gel Tech, 2002) describes a method of preparing a sunscreen composition with improved photostability that contains at least two sunscreen active ingredients, which are photo-unstable when formulated together, which method comprises the steps of separating the two ingredients from each other by microencapulating at least one of said ingredients in sol-gel microcapsules.

While some methods of enhancing the stability of organic sunscreens have been reported, there exists a need to develop compositions that provide enhanced stability so that the skin is protected against the harmful effects of UV radiation for longer and longer periods of time. The present inventors have during the course of intensive research into many different methods of preparing sunscreen composition where the organic sunscreens are kept stable, developed a complex of a selected class of metal ions with an organic sunscreen that solves most of the problems of the prior art sunscreen compositions.

U.S. Pat. No. 6,419,907 (L'Oreal, 2002) describes a cosmetic composition for protecting the skin against ultra-violet radiation comprising a cosmetic vehicle containing at least one fatty phase, an effective amount of copper (II) bis(3,5-diisopropylsalicylate) and optionally one or more of a UV-B or a UV-A sunscreen.

WO 93/11095 (Richardson Vicks) describes a sunscreen complex having a UV-A absorbing chromophore, a UV-B absorbing chromophore and a metal cation. The metal ion could be selected from a long exhaustive list of metal cations.

Although the above two prior art documents deal with some metal compounds in combination with sunscreens or metal complexes with sunscreens, it does not teach that a selective list of metals ions when complexed with organic sunscreens provides for vastly improved stability in cosmetic compositions.

It is thus an object of the invention to provide a stable sunscreen composition.

It is another object of the invention to provide a stable sunscreen composition that contains both UV-A and UV-B sunscreens, which conventionally are known to interact with each other and have low stability in the composition.

It is another object of the invention to provide a stable sunscreen composition that can be prepared by a simple and cost-effective process.

### SUMMARY OF THE INVENTION

According to the first aspect of the invention there is provided a stable sunscreen composition comprising:

(a) a complex of a first organic sunscreen with a first paramagnetic metal ion; and

(b) a second organic sunscreen.

Particularly preferred paramagnetic metal ions are Cu²⁺, Fe³⁺, Co²⁺, Mn²⁺ or Ti³⁺.

Particularly preferred organic sunscreens are 4-t-butyl-4′-methoxydibenzoylmethane or 4-methoxycinnamate.

According to a second aspect of the invention there is provided a process for preparation of the stable sunscreen composition of the first aspect wherein complex of the organic sunscreen with a paramagnetic metal ion is prepared using a process comprising the steps of:
(a) dissolving/dispersing an organic sunscreen in a solvent to prepare a solution/dispersion
(b) treating said solution/dispersion with an alkaline material; and
(c) treating the product of step (b) with a salt of said paramagnetic metal ion.

### DETAILED DESCRIPTION OF THE INVENTION

The invention provides for a stable sunscreen composition containing an organic sunscreen. The stable sunscreen composition of the invention comprises a complex of a first organic sunscreen with a first paramagnetic metal ion and a second organic sunscreen. The word “complex” is meant a co-ordination complex where the metal ion is linked to the donor atom, such as oxygen and nitrogen present in the organic sunscreen, by a co-ordinate covalent bond.

The second organic sunscreen may be complexed to a second paramagnetic metal ion thereby to form a second complex with the proviso that the first and second organic sunscreens and the first and second paramagnetic metal ions are not the same.

According to a preferred aspect of the present invention the sunscreen composition comprises a complex of paramagnetic metal ion with a dibenzoylmethane derivative and a complex of paramagnetic metal ion with a methoxycinnamate derivative.

A ligand of the first sunscreen and a ligand of the second organic sunscreen may be complexed to the same paramagnetic metal ion.

The paramagnetic metal ions useful in the present invention are Cu²⁺, Fe³⁺, Co²⁺, Mn²⁺, Ti³⁺, V²⁺, V⁴⁺, Cr²⁺, Cr³⁺, Cr⁴⁺, Mn³⁺, Mn⁴⁺, Ni²⁺, Ni³⁺, Mo³⁺, Tc⁶⁺, R³⁺, R⁴⁺, Ce³⁺, Pr³⁺, Nd³⁺, Pm³⁺, Sm³⁺, Eu³⁺, Gd³⁺, Tb³⁺, Tb⁴⁺, Dy³⁺, Ho³⁺, Er³⁺, Tm³⁺, Yb³⁺, R²⁺, R⁴⁺, R⁶⁺, Os³⁺, Os⁴⁺, Os⁶⁺, Ir³⁺, Ir⁴⁺, Ir⁶⁺, Au³⁺ and Hg⁺. Preferred paramagnetic metal ions are those from the transition metal or inner transition metal groups i.e ‘d’ block or ‘f’ block metals. More preferred paramagnetic metal ions are selected from Cu²⁺, Fe³⁺, Co²⁺, Mn²⁺ or Ti³⁺, more preferably Cu²⁺, Fe³⁺ or Mn²⁺.

A complex of any organic sunscreen complex with a paramagnetic metal ion is suitable for use in the compositions of the present invention. Preferred sunscreens are compounds containing cycloalkyl, aryl, hydroxyl, carbonyl, carboxylic or sulphonic acid functional groups. Suitable organic sunscreens include 2-hydroxy-4-methoxybenzophenone, octyidimethyl-p-aminobenzoic acid, digalloyltrioleate, 2,2-dihydroxy-4-methoxybenzophenone, ethyl+(bis(hydroxypropyl)) aminobenzoate, 2-ethylhexyl-2-cyano-3,3-diphenylacrylate, 2-ethylhexylsalicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butyl methoxydibenzoylmethane, 2-hydroxy-4-methoxybenzophenone, octyidimethyl-p-aminobenzoic acid and mixtures thereof. Further preferred are sunscreens which are derivatives of □-diketone or carboxylic acid functional groups having an absorbance maximum in the range of 250-450 nm. Further more preferred sunscreens are dibenzoylmethane or cinnamic acid or derivatives thereof.

The UV-A sunscreen is preferably a dibenzoylmethane derivative. The most preferred dibenzoyl methane derivative is 4-t-butyl-4′-methoxydibenzoylmethane which is available under the brand name Parsol 1789 from Givaudan. Other dibenzoylmethane derivatives which may be included in the composition of the invention include 2,4-dimethyl-4′-methoxydibenzoylmethane, 2-methyl-5-t-butyl-4′-methoxydibenzoylmethane, 4,4′-diisopropyl-dibenzoylmethane and 2,4-dimethyl-dibenzoylmethane. The preferred UV-B sunscreen which is complexed with a paramagnetic metal ion is 4-methoxycinnamate.

Most suitable organic sunscreens are 4-methoxycinnamate and 4-t-butyl-4-methoxydibenzoylmethane.

The non-complexed organic sunscreen may be selected from any one of the organic sunscreens set forth hereinabove.

The said complex is preferably present in an amount in the range of 0.1% to 15% by weight of the composition. The preferred amounts of the complex of paramagnetic metal ion with dibenzoylmethane derivative in the sunscreen composition of the present invention is in the range of 0.1 to 6% by weight of the composition. The preferred amounts of the complex of paramagnetic metal ion with methoxycinnamate derivative in the sunscreen composition of the present invention is in the range of 0.1 to 13% by weight of the composition.

Useful inorganic sunblocks are also preferably used in the present invention. These include, for example, zinc oxide, iron oxide, silica, such as fumed silica, and titanium dioxide.

Ultrafine titanium dioxide in either of its two forms, namely water-dispersible titanium dioxide and oil-dispersible titanium dioxide is especially suitable for the invention. Water-dispersible titanium dioxide is ultrafine titanium dioxide, the particles of which are non-coated or which are coated with a material to impart a hydrophilic surface property to the particles. Examples of such materials include aluminium oxide and aluminium silicate.

Oil-dispersible titanium dioxide is ultrafine titanium dioxide, the particles of which exhibit a hydrophobic surface property, and which, for this purpose, can be coated with metal soaps such as aluminium stearate, aluminium laurate or zinc stearate, or with organosilicone compounds.

By “ultrafine titanium dioxide” is meant particles of titanium dioxide having an average particle size of less than 100 nm, preferably 70 nm or less; more preferably from 10 to 40 nm and most preferably from 15 to 25 nm.

By topical application to the skin of a mixture of both water-dispersible ultrafine-titanium dioxide and oil-dispersible ultrafine titanium dioxide, synergistically enhanced protection of the skin against the harmful effects of both UV-A and UV-B rays is achievable.

Ultrafine titanium dioxide is the preferred inorganic sunblock agent as per this invention. The total amount of sun block that is preferably incorporated in the composition according to the invention is from 0.1 to 5% by weight of the composition.

The sunscreen composition preferably comprises a skin-lightening agent. The skin lightening agent is preferably chosen from one or more of a vitamin B3 compound or its derivatives e.g. niacin, nicotinic acid, niacinamide or other well known skin lightening agents e.g. adapalene, aloe extract, ammonium lactate, anethole derivatives, apple extract, arbutin, azelaic acid, kojic acid, bamboo extract, bearberry extract, bletilla tuber, bupleurum falcatum extract, burnet extract, butyl hydroxy anisole, butyl hydroxy toluene, citrate esters, Chuanxiong, Dang-Gui, deoxyarbutin, 1,3-diphenylpropane derivatives, 2,5-dihydroxybenzoic acid and its derivatives, 2-(4-acetoxyphenyl)-1,3-dithane, 2-(4-hydroxyphenyl)-1,3-dithane, ellagic acid, escinol, estragole derivatives, Fadeout (available from Pentapharm), Fangfeng, fennel extract, ganoderma extract, gaoben, Gatuline Whitening (available from Gattlefosse), genistic acid and its derivatives, glabridin and its derivatives, gluco pyranosyl-1-ascorbate, gluconic acid, glycolic acid, green tea extract, 4-hydroxy-5-methyl-3[2H]-furanone, hydroquinone, 4-hydroxyanisole and its derivatives, 4-hydroxybenzoic acid derivatives, hydroxycaprylic acid, inositol ascorbate, kojic acid, lactic acid, lemon extract, linoleic acid, magnesium ascorbyl phosphate, Melawhite (available from Pentapharm), morus alba extract, mulberry root extract, 5-octanoyl salicylic acid, parsley extract, phellinus linteus extract, pyrogallol derivatives, 2,4-resorcinol derivatives, 3,5-resorcinol derivatives, rose fruit extract, salicylic acid, Song-Yi extract, 3,4,5 trihydroxybenzyl derivatives, tranexamic acid, vitamins like vitamin B6, vitamin B12, vitamin C, vitamin A, dicarboxylic acids, resorcinol derivatives, extracts from plants viz. rubia and symplocos, hydroxycarboxylic acids like lactic acid and their salts e.g. sodium lactate, and mixtures thereof. Vitamin B3 compound or its derivative e.g. niacin, nicotinic acid, niacinamide are the more preferred skin lightening agent as per the invention, most preferred being niacinamide. Niacinamide, when used, is preferably present in an amount in the range of 0.1 to 10%, more preferably 0.2 to 5% by weight of the composition.

Cosmetic compositions to deliver different benefit agents are prepared using different cosmetically acceptable emulsifying systems and vehicles. For the present invention, a cosmetically acceptable base comprises 5 to 25% fatty acid or 0.1 to 80% soap. Mixtures of fatty acid and soap are also suitable e.g. vanishing cream base which gives a highly appreciated matty feel to the skin. C₁₂ to C₂₀ fatty acids are especially preferred for the present invention, more preferred being C₁₄ to C₁₈ fatty acids. The most preferred fatty acid is stearic acid. The fatty acid in the composition is more preferably present in an amount in the range of 5 to 20% by weight of the composition. Soaps in the vanishing cream base include alkali metal salt of fatty acids, like sodium or potassium salts, most preferred being potassium stearate. The soap in the vanishing cream base is generally present in an amount in the range of 0.1 to 10%, more preferably 0.1 to 3% by weight of the composition. Generally the vanishing cream base in cosmetic compositions is prepared by taking a desired amount of total fatty matter and mixing with potassium hydroxide in desired amounts. The soap is usually formed in situ during the mixing. The sunscreen composition usually comprises greater than 50% water.

Alternately the skin lightening composition is a wash-off product e.g. soap having 5 to 80% salt of fatty acids.

Optional Ingredients

The composition according to the invention may also comprise other diluents. The diluents act as a dispersant or carrier for other materials present in the composition, so as to facilitate their distribution when the composition is applied to the skin.

Diluents other than water can include liquid or solid emollients, solvents, humectants, thickeners and powders. Examples of each of these types of vehicle, which can be used singly or as mixtures of one or more vehicles, are as follows:

Emollients such as stearyl alcohol, glyceryl monoricinoleate, mink oil, cetyl alcohol, isopropyl isostearate, stearic acid, isobutyl palpitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone bils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate and myristyl myristate;

Propellants such as propane, butane, isobutane, dimethyl ether, carbon dioxide, nitrous oxide;

Solvents such as ethyl alcohol, isopropanol, acetone, ethylene glycol monoethyl ether, diethylene glycol monobutyl ether and diethylene glycol monoethyl ether; and

Powders such as chalk, talc, Fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

These additional materials are preferably present from 10 to 99.9%, preferably from 50 to 99% by weight of the cosmetic composition, and can, in the absence of other cosmetic adjuncts, form the balance of the composition.

Optional Skin Benefit Agents:

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include: antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents and skin healing agents.

According to a second aspect of the invention, there is provided a sunscreen composition of the first aspect wherein the complex of organic sunscreen with paramagnetic metal ion is prepared using a process comprising the steps of:
(a) dissolving/dispersing said organic sunscreen in a solvent to prepare a solution/dispersion;
(b) treating said solution/dispersion with an alkaline material; and
(c) treating the product of step (b) with a salt of said paramagnetic metal ion.

The solvent may be selected from any organic solvent e.g. ethanol, methanol, propanol, isopropyl alcohol, butyl alcohol, tetrahydrofuran, acetone, dichloromethane, chloroform, toluene, dimethyl sulfoxide, dimethylformamide and benzene, the preferred solvents being ethanol or methanol. The alkaline material may be sodium hydroxide, potassium hydroxide, lithium hydroxide, ammonium hydroxide, sodium acetate, potassium t-butoxide, sodium ethoxide, sodamide, aniline, diphenyl amine, N-methylaniline, pyridine and ethylenediamine, the preferred alkaline material being sodium hydroxide, sodium acetate or ammonium hydroxide. Usually the complex of the organic sunscreen with the paramagnetic metal ion precipitates after step (iii) of the process. The precipitate is then usually washed and dried to prepare a powder of the complex. The powder may then be used in the sunscreen composition of the invention.

The invention will now be illustrated with reference to the following non-limiting examples.

### EXAMPLES

### Comparative Examples A-D

Comparative example A was a control sample of Parsol 1789. Comparative examples B to D were complexes of Parsol 1789 with a diamagnetic metal ion e.g. Co³⁺ (comparative example B), Zn²⁺ (comparative example C) and Al³⁺ (comparative example D).

### Examples 1-3

Examples 1-3 were complexes of Parsol 1789 with paramagnetic metal ion. In example 1 the paramagnetic metal ion was Cu²⁺, in example 2 it was Co²⁺, while in example 3 it was Fe³⁺.

The complex of Parsol 1789 with metal ions were prepared by the method given below.

Method of Preparation of the Metal Complexes:

Parsol 1789 was taken in methanol in a 250 ml round bottom flask. An equimolar quantity of sodium acetate was added to Parsol 1789 under continuous stirring. A methanolic solution of the required salt (typically metal chloride) in the required molar ratio (metal:Parsol 1789=1:2 for Cu(II), Zn(II) and Co(II); 1:3 for Fe(III), Co(III) and AI(III) was added to the mixture. This reaction mixture was stirred for about an hour when the complex precipitated out. The precipitate was filtered by suction, washed thoroughly with methanol and dried in air.

The Parsol 1789/Parsol 1789 complexes were tested using test procedure 1 given below.

Test Procedure 1

Parsol 1789 and its metal complexes were dissolved in dichloromethane and diluted to an absorbance of 1. Petri dishes on which sunscreen are deposited were prepared by evaporating 25 ml of the above solution on a Petri dish in the dark at room temperature. Several such Petri dishes were prepared. The dried Petri dishes were exposed to mid-noon sunlight and one Petri dish was removed after every 20 minutes of exposure. A blank was also carried out wherein the Petri dish was not exposed to sunlight. After sun exposure, the contents in the Petri dish were redissolved in dichloromethane and made up to 25 ml. The absorbance of these solutions was measured. The percentage of the sunscreen in the blank was taken to be 1.00% and the percentage of sunscreen in the exposed samples were determined relative to the blank using formula (I) given below:

$${Q}_{t} = \frac{{A}_{t}}{{A}_{0}} \times 100 \qquad ( 1 )$$

where Qₜ is the percentage of sunscreen present after t minutes of sun exposure, A₀ and Aₜ are the absorbance of sunscreen solutions after sun exposure for 0 minutes and t minutes.

The results of the stability of Parsol 1789 and the various complexes are given in table 1.

TABLE 1

| Sample | 0 mins | 20 mins | 40 mins | 60 mins |
|---|---|---|---|---|
| Comp. exp A | 100 | 71.6 | 61.1 | 41.9 |
| Comp. exp B | 100 | 76.9 | 66.6 | 59.1 |
| Comp. exp C | 100 | 22.4 | 17.3 | 13.2 |
| Comp. exp D | 100 | 67.2 | 47.4 | 27.0 |
| Example 1 | 100 | 97.1 | 98.3 | 96.7 |
| Example 2 | 100 | 94.4 | 93.1 | 92.1 |
| Example 3 | 100 | 98.2 | 96.7 | 94.6 |

The above table indicates that a complex of an organic sunscreen with a paramagnetic metal ion (examples 1-3) is far more stable: as compared to the sunscreen alone or complex with diamagnetic metal ions.

Sunscreen Compositions: Comparative Example E and Example 4

Sunscreen compositions as given in table 2 below were prepared. Comparative example E is a conventional sunscreen cream having Parsol 1789 and Parsol MCX. Example 4 is a sunscreen cream of the invention comprising a complex of Parsol 1789 with a paramagnetic metal ion Cu²⁺.

TABLE 2

| Components | Comp. ex. E | Example 4 |
|---|---|---|
| Stearic acid | 18 | 18 |
| Niacinamide | 1 | 1 |
| Potassium hydroxide (85%) | 0.6 | 0.6 |
| Cetyl alcohol | 0.5 | 0.5 |
| Silicone oil DC 200 | 0.5 | 0.5 |
| Isopropyl myristate | 0.75 | 0.75 |
| Methyl/propyl paraben | 0.3 | 0.3 |
| Titanium dioxide | 0.20 | 0.20 |
| Parsol 1789 | 0.4 | — |
| Cu(Parsol 1789)₂ | — | 0.4 |
| Parsol MCX | 0.75 | 0.75 |
| Water | To 100 | To 100 |

The samples were tested for stability using test procedure 2 set forth hereinbelow and the results are summarized in table 3 and table 4.

Test Procedure 2

10 mg of the cream was spread on a two square centimetre area on a glass plate. Several such glass slides were prepared. The slides were exposed to midnoon sunlight. After every 20 minutes of sun exposure, a slide was removed from sunlight. A blank experiment was also carried out in which the slide was not exposed to sunlight. The creams in the exposed and blank slides were extracted individually in dichloromethane, made up to 25 ml in a volumetric flask and their absorbance measured using a Perkin-Elmer spectrophotometer. From the absorbance, the amount of sunscreen remaining was calculated. The sunscreen in blank slide was taken to be 100% and all others were quantified with respect to it using equation (1) given above.

Table 3 shows the percentage of Parsol 1789/Parsol 1789 metal complex remaining after exposure of the sample to sunlight after a specified amount of time following test procedure 2.

TABLE 3

| Sample | 0 mins | 20 mins | 40 mins | 60 mins |
|---|---|---|---|---|
| Comparative | 100 | 44 | 34 | 17 |
| example E | | | | |
| Example 4 | 100 | 100 | 90 | 83 |

Thus it can be seen that the percentage of Parsol 1789 remaining is significantly higher in Example 4 than in Example E, even after 60 minutes.

Table 4 shows the percentage of Parsol MCX remaining after exposure of the sample to sunlight after a specified amount of time following test procedure 2.

TABLE 4

| Sample | 0 mins | 20 mins | 40 mins | 60 mins |
|---|---|---|---|---|
| Comparative | 100 | 56 | 42 | 31 |
| example E | | | | |
| Example 4 | 100 | 91 | 70 | 63 |

Thus it can be seen that the percentage of Parsol MCX remaining is significantly higher in Example 4 than in Example E, even after 60 minutes.

In another set of experiments, the photostability of a mixture of Parsol 1789 and Parsol MCX was studied in complexed and un-complexed states. While the copper complex of Parsol 1789 was prepared by the procedure mentioned hereinabove, the copper complex of Parsol MCX was prepared by the following procedure.

Procedure

Parsol MCX was taken in methanol in a 250 ml round bottom flask. An equimolar quantity of sodium acetate was added to the Parsol MCX under continuous stirring. A methanolic solution of copper chloride in molar ratio (metal:Parsol MCX) of 1:2 was added to the methanolic solution. This reaction mixture was stirred for about one hour when the complex precipitated out. The precipitate was filtered by suction, washed thoroughly with methanol and dried in air.

Test Procedure 3

The photostability of the mixture of Parsol 1789 and Parsol MCX was studied by dissolving the mixture in 25 ml dichloromethane, such that its absorbance is close to 1. The λₘₐₓ for 4-methoxy cinnamic acid (Parsol MCX) is approximately 310 nm and the λₘₐₓ for Parsol 1789 is approximately 360 nm. The solution was poured into a Petri dish and allowed to evaporate in the dark at room temperature (25 degrees Centigrade). Five such petridishes were prepared for each solution. Four Petri dishes were exposed to mid-noon sunlight and one Petri dish was removed every 15 minutes for further analysis. One Petri dish containing evaporated sunscreen mixture was not exposed to sunlight and was considered as the control. After sun exposure, the contents in the Petri dish were re-dissolved in dichloromethane and the volume was made up to 25 ml. The absorbance of each of these solutions was measured at the λₘₐₓ values mentioned above. The percentage of the sunscreen in the control was taken to be 100, and the percentage of sunscreen in the exposed samples were determined relative to the control using formula (1) given above.

The observations of the relative stability of complexed and uncomplexed Parsol 1789 and that of Parsol MCX are given in tables 5 and 6 below.

TABLE 5
UV-A photostability at 360 nm

| | % of sunscreen | | | | |
|---|---|---|---|---|---|
| | at time (min) | | | | |
| Mixture | 0 | 15 | 30 | 45 | 60 |
| Parsol 1789 + Parsol MCX (both | 100 | 69 | 37 | 20 | 10 |
| un-complexed) | | | | | |
| Copper(Parsol 1789)₂ complex + Parsol | 100 | 95 | 92 | 90 | 87 |
| MCX | | | | | |
| Parsol 1789 + Copper(Parsol MCX)₂ | 100 | 100 | 96 | 83 | 78 |
| complex | | | | | |
| Copper(Parsol 1789)₂ complex + | 100 | 100 | 98 | 94 | 94 |
| Copper(Parsol MCX)₂ complex | | | | | |

TABLE 6
UV-B photostability at 310 nm

| | % of sunscreen | | | | |
|---|---|---|---|---|---|
| | at Time (min) | | | | |
| Mixture | 0 | 15 | 30 | 45 | 60 |
| Parsol 1789 + Parsol MCX (both | 100 | 88 | 66 | 52 | 49 |
| un-complexed) | | | | | |
| Copper(Parsol 1789)₂ complex + Parsol MCX | 100 | 89 | 74 | 73 | 56 |
| Parsol 1789 + Copper(Parsol MCX)₂ complex | 100 | 72 | 69 | 59 | 58 |
| Copper(Parsol 1789)₂ complex + | 100 | 97 | 92 | 80 | 77 |
| Copper(Parsol MCX)₂ complex | | | | | |

Thus, it will be readily apparent that the photostability of Parsol MCX and Parsol 1789 is substantially improved in their complexed forms, as compared to uncomplexed forms, and it remains high even after 60 minutes of sun exposure.

Stability was also measured in cosmetic compositions. The procedure adopted was the same as set forth herinabove in Test Procedure 2 except that a 1:2 mixture (by volume) of methanol and dichloromethane was substituted for dichloromethane for solution preparation and extraction. The reason for this is that stearic acid is insoluble in dichloromethane (but soluble in methanol) and copper complexes of Parsol 1789 and Parsol MCX are soluble in dichloromethane but not in methanol. Instead of withdrawing the Petri dishes at 20 minutes as in Test Procedure 2, they were withdrawn at 15 minute intervals.

The three compositions in table 7 below were prepared using the standard procedure of making vanishing creams.

TABLE 7

| | Weight (g) | | |
|---|---|---|---|
| | Comparative. | | |
| Components | example F | Example 5 | Example 6 |
| Stearic acid | 18 | 18 | 18 |
| Niacinamide | 1 | 1 | 1 |
| Potassium hydroxide (85%) | 0.6 | 0.6 | 0.6 |
| Cetyl alcohol | 0.5 | 0.5 | 0.5 |
| Silicone oil DC 200 | 0.5 | 0.5 | 0.5 |
| Isopropyl myristate | 0.75 | 0.75 | 0.75 |
| Methyl/propyl paraben | 0.3 | 0.3 | 0.3 |
| Titanium dioxide | 0.20 | 0.20 | 0.20 |
| Parsol 1789 | 0.4 | — | 0.4 |
| Copper(Parsol 1789)₂ complex | — | 0.4 | |
| Parsol MCX | 0.75 | 0.75 | |
| Copper(Parsol MCX)₂ complex | | | 0.75 |
| Water | To 100 | To 100 | To 100 |

The results are presented in tables 8 and 9 below.

TABLE 8
UV-A photostability at 360 nm

| | % of sunscreen | | | | |
|---|---|---|---|---|---|
| | at time (min) | | | | |
| Cream compositions containing | 0 | 15 | 30 | 45 | 60 |
| Parsol 1789 + Parsol MCX (both | 100 | 46 | 31 | 18 | 13 |
| un-complexed) Comparative Example F | | | | | |
| Copper(Parsol 1789)₂ complex + Parsol MCX | 100 | 96 | 93 | 91 | 87 |
| Example 5 | | | | | |
| Parsol 1789 + Copper(Parsol MCX)₂ complex | 100 | 96 | 89 | 85 | 78 |
| Example 6 | | | | | |

TABLE 9
UV-B photostability at 310 nm

| | % of sunscreen | | | | |
|---|---|---|---|---|---|
| | at Time (min) | | | | |
| Cream compositions containing | 0 | 15 | 30 | 45 | 60 |
| Parsol 1789 + Parsol MCX (both | 100 | 58 | 52 | 40 | 35 |
| un-complexed) Comparative Example F | | | | | |
| Copper(Parsol 1789)₂ complex + Parsol MCX | 100 | 84 | 72 | 60 | 41 |
| Example 5 | | | | | |
| Parsol 1789 + Copper(Parsol MCX)₂ complex | 100 | 81 | 72 | 68 | 62 |
| Example 6 | | | | | |

It can be readily seen from the tables 8 and 9 that the stability of sunscreens is vastly improved when at least one of the sunscreens is present in the form of a complex with a paramagnetic metal ion, even at the end of 60 minutes.

The illustrated examples thus provide for a sunscreen composition having enhanced stability as compared to that reported in the prior art.

1. A sunscreen composition comprising
(a) a complex of a first organic sunscreen with a first paramagnetic metal ion; and
(b) a second organic sunscreen.

2. A sunscreen composition according to claim 1 wherein the second organic sunscreen is complexed to a second paramagnetic metal ion thereby to form a second complex with the proviso that the first and second organic sunscreens and the first and second paramagnetic metal ions are not the same.

3. A sunscreen composition according to claim 2 wherein a ligand of the first sunscreen and a ligand of the second organic sunscreen are complexed to the same paramagnetic metal ion.

4. A sunscreen composition as claimed in claim 1, wherein said organic sunscreen is dibenzoylmethane or cinnamic acid or derivatives thereof.

5. A sunscreen composition as claimed in claim 1, wherein the paramagnetic metal ion is selected from Cu²⁺, Fe³⁺, Co²⁺, Mn²⁺ or Ti³⁺.

6. A sunscreen composition as claimed in claim 5 wherein said paramagnetic metal ion is Cu²⁺, Fe³⁺ or Mn²⁺.

7. A sunscreen composition as claimed in claim 4 when dependent on claim 2 comprising a complex of a paramagnetic metal ion with a derivative of dibenzoylmethane and a complex of a paramagnetic metal ion with a derivative of cinnamic acid.

8. A sunscreen composition as claimed in claim 4, wherein said derivative of dibenzoylmethane is 4-t-butyl-4′-methoxydibenzoylmethane.

9. A sunscreen composition as claimed in claim 4, wherein said derivative of cinnamic acid is 4-methoxycinnamate.

10. A sunscreen composition as claimed in claim 4, wherein said complex of a derivative of dibenzoylmethane is present in an amount in the range of 0.1% to 6% by weight of the composition.

11. A sunscreen composition as claimed in claim 4, wherein said complex of a derivative of cinnamic acid is present in an amount in the range of 0.1% to 13% by weight of the composition.

12. A sunscreen composition as claimed in claim 1, comprising a skin-lightening agent.

13. A sunscreen composition as claimed in claim 12 wherein said skin-lightening agent is niacinamide.

14. A sunscreen composition as claimed in claim 13 wherein niacinamide is present in an amount in range of 0.1% to 10% by weight of the composition.

15. A sunscreen composition as claimed in claim 1, comprising 5% to 25% by weight C₁₂-C₂₀ fatty acids and 0.1% to 10% by weight fatty acid soap.

16. A sunscreen composition as claimed in claim 1, wherein said composition comprises at least 50% by weight water.

17. A sunscreen composition as claimed in claim 1, wherein said complex of organic sunscreen with a paramagnetic metal ion is prepared using a process comprising the steps of:
(a) dissolving/dispersing said organic sunscreen in a solvent to prepare a solution/dispersion;
(b) treating said solution/dispersion with an alkaline material; and
(c) treating the product of step (b) with a salt of said paramagnetic metal ion.

18. A sunscreen composition as claimed in claim 17 wherein said solvent is ethanol or methanol.

19. A sunscreen composition as claimed in claim 17 wherein said alkaline material is sodium hydroxide, sodium acetate or ammonium hydroxide.
